(19) United States
(12) Patent Application Publication
(10) Pub. No.: US 2026/0013909 A1
(43) Pub. Date: Jan. 15, 2026

(54) DEVICES AND METHODS FOR SACROILIAC JOINT FUSING

(71) Applicant: KIC VENTURES, LLC, BURLINGTON, MA (US)

(72) Inventors: KINGSLEY R. CHIN, BURLINGTON, MA (US); VITO LORE, BURLINGTON, MA (US); JOSHUA FINKEL-LOPEZ, BURLINGTON, MA (US)

(73) Assignee: KIC VENTURES, LLC, BURLINGTON, MA (US)

(21) Appl. No.: 19/262,175

(22) Filed: Jul. 8, 2025

Related U.S. Application Data
(60) Provisional application No. 63/670,534, filed on Jul. 12, 2024.

(51) Int. Cl. A61B 17/70; A61B 17/00; A61B 17/16; A61B 90/00

(52) U.S. Cl. CPC A61B 17/7055; A61B 17/1615; A61B 17/1671; A61B 90/06; A61B 90/08; A61B 2017/00017; A61B 2017/00681; A61B 2017/00743; A61B 2017/00982; A61B 2090/061; A61B 2090/0807

(57) ABSTRACT

An awl-tap-dilator tool used in a single step surgical technique, includes an elongated shaft having a beveled tip at a distal end, a drill-tap section adjacent to the beveled tip, and a dilator section adjacent to the drill-tap section. The beveled tip is used for piercing a bone and locate an appropriate trajectory under fluoroscopy. The drill-tap section is used for drilling and tapping the bone in order to form a bone opening. The dilator section surrounds the elongated shaft, has a larger diameter than the elongated shaft, and is used for dilating tissue around and above the bone opening.

### CROSS REFERENCE TO RELATED CO-PENDING APPLICATIONS

This application claims the benefit of U.S. provisional application Ser. No. 63/668,681 filed Jul. 12, 2024 and entitled “DEVICES AND METHODS FOR SACROILIAC JOINT FUSING”, the contents of which are expressly incorporated herein by reference.

### FIELD OF THE INVENTION

The present invention relates to devices and methods for sacroiliac (SI) joint fusing, and in particular, to devices, instruments, and methods that limit the size of the incision, and utilize a single step and single use surgical technique and instrument for accurate targeting and fusing of the SI joint.

### BACKGROUND OF THE INVENTION

The human spine includes individual vertebras that are connected to each other. Under normal circumstances the structures that make up the spine function are configured to protect the neural structures, allow us to stand erect, bear axial loads, and are flexible for bending and rotation. Disorders of the spine occur when one or more of these spine structures are abnormal. In these pathologic circumstances, surgery may be tried to restore the spine to the normal state and to relieve the patient of pain. Spine surgery for a multitude of spinal disorders is often used for filling voids within a pathologic vertebral body (exemplified by kyphoplasty or vertebroplasty procedures), replacement of a degenerated intervertebral disc with an intervertebral implant device that preserves mobility (disc replacement) or for fusing adjacent vertebral segments (interbody and posterolateral fusions). Fusion works well because it stops pain due to movement at the joints, holds the spine in place after correcting a deformity, and prevents instability and or deformity of the spine after spine procedures such as laminectomies or verterbrectomies.

One area where fusion is applicable is for sacroiliac joint fusion (SIJF). The sacroiliac joint (SIJ) 50 is a firm, small joint that lies at the junction of the sacrum 25 and the pelvis 20, as shown in FIG. 1. While most of the vertebras 30 of the spine are mobile, the sacrum 25 is made up of five vertebras that are fused together and do not move. The iliac bones 20a, 20b are the two large bones that make up the pelvis 20. The sacroiliac joints 50 connect the spine 10 to the pelvis 20. The sacrum 25 and the iliac bones 20a, 20b are held together by a collection of strong ligaments. These joints are important in transferring the load of the upper body to the lower body, supporting the entire weight of the upper body when we are erect, which in turn results in stress to this weight-bearing area of the pelvis and spine. Pathologies of the SIJ include degenerative sacroiliitis (arthritis), sacroiliac disruption, tumors and other type of injuries. Sacroiliac joint (SI) fusion is used for treating degenerative sacroiliitis, sacroiliac disruption, and for stabilizing the SI joint after sacrectomy or after injury.

There is increasing consensus among surgeons that there is a need to develop devices, instruments, and methods to limit the size of the incision, extensive muscle stripping, prolonged retraction of muscles for visualization, avoidance of neural tissue retraction and injury, and denervation and devascularization that are known to contribute to poorer patient outcome after traditional open surgeries to treat pathologies deep within the body. In many cases these complications lead to permanent scarring and pain that can be more severe than the pain from the initial ailment. Limiting these complications in addition to the operative, general anesthesia, and recovery times are among the goals of this invention and that of percutaneous or minimally invasive surgeries.

This invention addresses the need for bone fusing implants that are used for sacroiliac joint fusion and for implant insertion tools that adhere to the principals of the less exposure surgery (LES) of outpatient surgery, which include minimizing the size of the incision, minimizing extensive muscle stripping, minimizing prolonged retraction of muscles for visualization, and preventing neural tissue retraction and injury.

### SUMMARY OF THE INVENTION

The present invention relates to devices and methods for sacroiliac (SI) joint fusing, and in particular, to devices, instruments, and methods that limit the size of the incision, and utilize a single step and single use surgical technique and instrument for accurate targeting and fusing of the SI joint.

In general, in one aspect, the invention features an awl-tap-dilator tool used in a single step surgical technique, including an elongated shaft having a beveled tip at a distal end, a drill-tap section adjacent to the beveled tip, and a dilator section adjacent to the drill-tap section. The beveled tip is used for piercing a bone and locate an appropriate trajectory under fluoroscopy. The drill-tap section is used for drilling and tapping the bone in order to form a bone opening. The dilator section surrounds the elongated shaft and has a larger diameter than the elongated shaft.

Implementations of this aspect of the invention may include one or more of the following features. The elongated shaft further includes a handle at the proximal end of the elongated shaft and the handle is used for inserting and rotating the beveled tip and drill-tap section. The handle includes an arrow that functions as a directional marking indicating the beveled tip's orientation. The dilator section includes markings on an outer surface used for determining a length of an implant. The awl-tap-dilator tool further includes a tissue protector cannula that surrounds the dilator section. The tissue protector cannula includes teeth at a distal end, and the teeth are used for impacting the tissue protector into the bone around said bone opening. The tissue protector cannula includes a window at a proximal end and has a marking line that is used for aligning with one of the markings of the dilator section in order to determine a length of an implant. The dilator section is made of injection molded plastic, and the elongated shaft, the beveled tip, and the drill-tap section are made of stainless steel. The teeth of the tissue protector cannula are made of stainless steel and the tissue protector cannula is made of injection molded plastic. The awl-tap-dilator tool further includes a tissue protector impacting component. The tissue protector impacting component includes a flat surface at a proximal end and a side slot opening at a distal end. The side slot opening is shaped and sized to slide laterally over a proximal end of the dilator section while the flat surface of the proximal end of the tissue protector impacting component is used for advancing and impacting the tissue protector into the bone without changing position and direction of the awl-tap-dilator tool.

In general, in another aspect, the invention features a system used in a single step surgical technique for forming an opening into a bone and inserting an implant into the bone opening including an awl-tap-dilator tool, a tissue protector cannula, and an implant inserter tool. The awl-tap-dilator tool includes an elongated shaft having a beveled tip at a distal end, a drill-tap section adjacent to the beveled tip, and a dilator section adjacent to the drill-tap section. The beveled tip is used for piercing a bone and locate an appropriate trajectory under fluoroscopy. The drill-tap section is used for drilling and tapping the bone in order to form the bone opening, and the dilator section surrounds and has a larger diameter than the elongated shaft. The tissue protector cannula surrounds the dilator section, and includes teeth at a distal end. The teeth are used for impacting the tissue protector into the bone around said bone opening. The implant inserter tool is used for inserting an implant into the bone opening through the tissue protector, and includes an elongated cannulated outer shaft and an inner stylet extending through the outer shaft.

Implementations of this aspect of the invention may include one or more of the following features. The inserter tool has a handle at a proximal end and the inner stylet has a rotatable knob at a proximal end. The inserter tool handle includes a center cutout that is shaped and sized to hold the rotatable knob. The inner stylet has a threaded distal end that is sized to engage threads on a proximal end of the implant. The system further includes a power adapter component used for attaching a power drill to the handle of the inserter tool. The power adapter component includes a shaft and a coupling member. The shaft has a proximal end shaped and sized to engage a power drill tip and the coupling member extends horizontally from a distal end of the shaft. A bottom surface of the coupling member includes pins that are shaped and sized to fit within and engage corresponding openings on a top surface of the handle.

In general, in another aspect, the invention features a method for forming an opening into a bone and inserting an implant into the bone opening in a single step include the following. First providing an awl-tap-dilator tool comprising an elongated shaft having a beveled tip at a distal end, a drill-tap section adjacent to the beveled tip, a dilator section adjacent to the drill-tap section, and a tissue protector cannula that surrounds the dilator section. Next, piercing the bone with the beveled tip and locating an appropriate trajectory under fluoroscopy. Next, drilling and tapping the bone with the drill-tap section and forming the bone opening. Next, dilating tissue above and around the bone opening with the dilator section, and then impacting the tissue protector cannula into the bone around the bone opening. Next, providing an implant inserter tool comprising an elongated cannulated outer shaft and an inner stylet extending through the outer shaft. Next, attaching the implant to a distal end of the inner stylet and inserting the implant inserter tool with the attached implant through the tissue protector, and then inserting the implant into the bone opening.

Implementations of this aspect of the invention may include one or more of the following features. The tissue protector cannula has a window at a proximal end and has a marking line that is used for aligning with a marking of the dilator section in order to determine a length of the implant. The tissue protector cannula has teeth at a distal end, and the teeth are used for impacting the tissue protector into the bone around the bone opening. The method may further include providing a tissue protector impacting component. The tissue protector impacting component includes a flat surface at a proximal end and a side slot opening at a distal end. The side slot opening is shaped and sized to slide laterally over a proximal end of the dilator section while the flat surface of the proximal end of the tissue protector impacting component is used for advancing and impacting the tissue protector into the bone without changing position and direction of the awl-tap-dilator tool.

The details of one or more embodiments of the invention are set forth in the accompanying drawings and description below. Other features, objects, and advantages of the invention will be apparent from the following description of the preferred embodiments, the drawings, and the claims.

### BRIEF DESCRIPTION OF THE DRAWINGS

Referring to the figures, wherein like numerals represent like parts throughout the several views:

FIG. 1A is a schematic posterior view of the pelvic bones and sacrum depicting;

FIG. 1B is a schematic posterior view of the pelvic bones and sacrum depicting four SIJ intra-articular bone openings;

FIG. 1C is a schematic side view of the pelvic bones and sacrum indicating a bone fusing implant as it is being inserted;

FIG. 1D is a schematic posterior view of the pelvic bones and sacrum depicting the inserted bone fusing implant into the intra-articular openings of FIG. 1B;

FIG. 2A is a side perspective view of the awl-tap-dilator tool, according to this invention;

FIG. 2B is a side perspective view of the awl-tap-dilator tool of FIG. 2A with the tissue protecting cannula;

FIG. 2C is a top view of the awl-tap-dilator tool with the tissue protecting cannula of FIG. 2B;

FIG. 2D is a top view of the awl-tap-dilator tool with the tissue protecting cannula of FIG. 2B and with the tissue protector impacting component, according to this invention;

FIG. 2E depicts an enlarged view of a window in the tissue protecting cannula of FIG. 2B;

FIG. 2F depicts a perspective side view of an inserter tool, according to this invention;

FIG. 2G depicts a perspective view of the distal end of the inserter tool of FIG. 2F with an attached implant, according to this invention;

FIG. 2H depicts a top perspective view of the inserter tool of FIG. 2F;

FIG. 2I depicts a perspective view of a power adapter component, according to this invention;

FIG. 2J depicts a top perspective view of the inserter tool of FIG. 2F with the attached power adapter component of FIG. 2I;

FIG. 2K depicts an enlarged top perspective view of the inserter tool of FIG. 2F;

FIG. 2L depicts a top perspective view of the inserter tool of FIG. 2F with an unlocking tool, according to this invention;

FIG. 2M depicts a top perspective view of the inserter tool of FIG. 2F with another embodiment of an unlocking tool, according to this invention;

FIG. 3A and FIG. 3B depict top and side views, respectively, of step (610) of the process 600 of targeting and fusing the SI joint of FIG. 4A;

FIG. 3C depicts a side view of step (620) of the process 600 of targeting and fusing the SI joint of FIG. 4A;

FIG. 3D depicts a side view of step (630) of the process 600 of targeting and fusing the SI joint of FIG. 4A;

FIG. 3E depicts a bottom view of step (640) of the process 600 of targeting and fusing the SI joint of FIG. 4A;

FIG. 3F depicts a side view of step (650) of the process 600 of targeting and fusing the SI joint of FIG. 4A;

FIG. 3G depicts a top view of step (660) of the process 600 of targeting and fusing the SI joint of FIG. 4A;

FIG. 3H depicts a side view of step (670) of the process 600 of targeting and fusing the SI joint of FIG. 4A;

FIG. 3I depicts a bottom view of step (680) of the process 600 of targeting and fusing the SI joint of FIG. 4B;

FIG. 3J depicts a top view of step (690) of the process 600 of targeting and fusing the SI joint of FIG. 4B;

FIG. 3K depicts a front view of step (695) of the process 600 of targeting and fusing the SI joint of FIG. 4B;

FIG. 3L is a schematic side view of the fused pelvic bone and sacrum with two inserted bone fusing implants; and

FIG. 4A and FIG. 4B depict a flow diagram of the process 600 of targeting and fusing the SI joint, according to this invention

### DETAILED DESCRIPTION OF THE INVENTION

The present invention relates to devices and methods for sacroiliac (SI) joint fusing, and in particular, to devices, instruments, and methods that limit the size of the incision, and utilize a single step and single use surgical technique and instrument for accurate targeting and fusing of the SI joint.

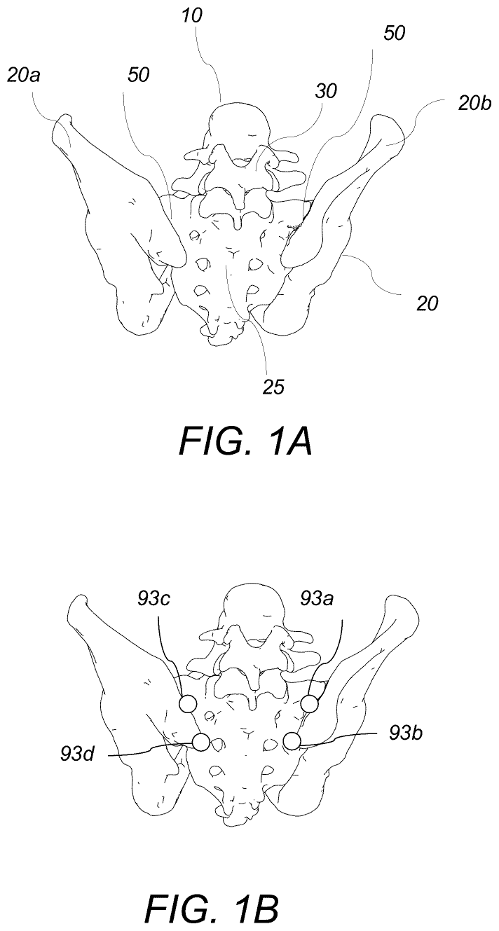

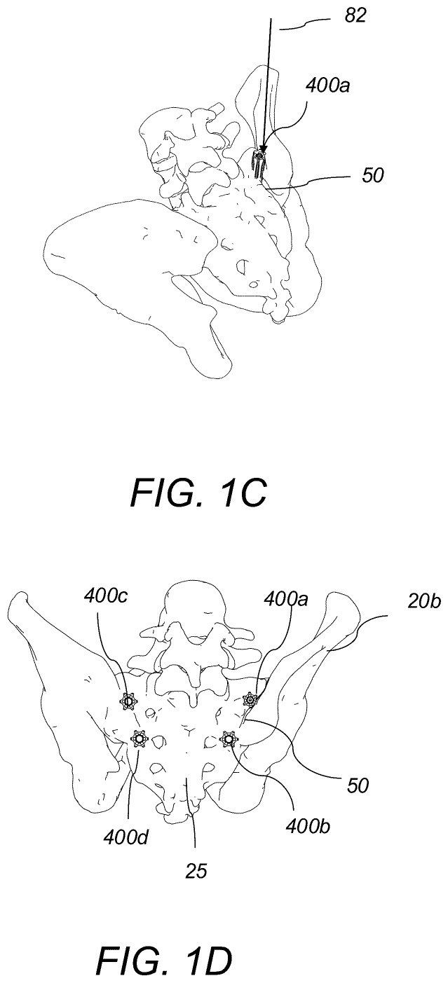

Referring to FIG. 1A-FIG. 1D, during sacroiliac joint fusion surgery, bone openings 93a-93d are formed into the joint lines 50 between the ilium bones 20a, 20b, and the adjacent sacrum 25 and then bone fusing implants 400a-400d are inserted into the bone openings 93a-93d along direction 82, as shown in FIG. 1C. During this surgery several tools are utilized including pins, pin guide tool, dilator, tissue protector, drill, and broach, among others, as described in U.S. Pat. No. 12,064,154B2, and U.S. Pat. No. 9,717,538B2, the contents of which are incorporated herewith by reference. In the present invention, a single step surgical technique and single use awl-tap-dilator tool 100 is used for accurate targeting and fusing of the SI joint.

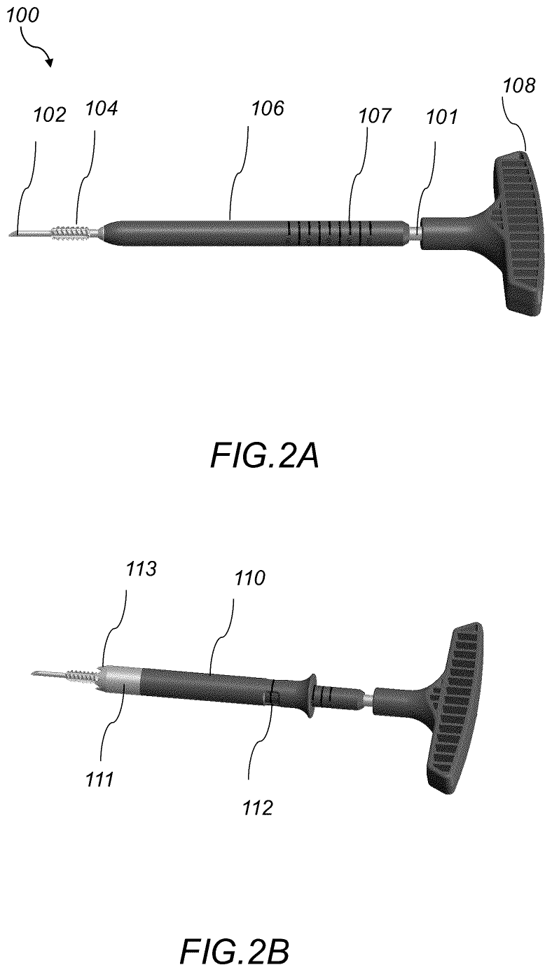

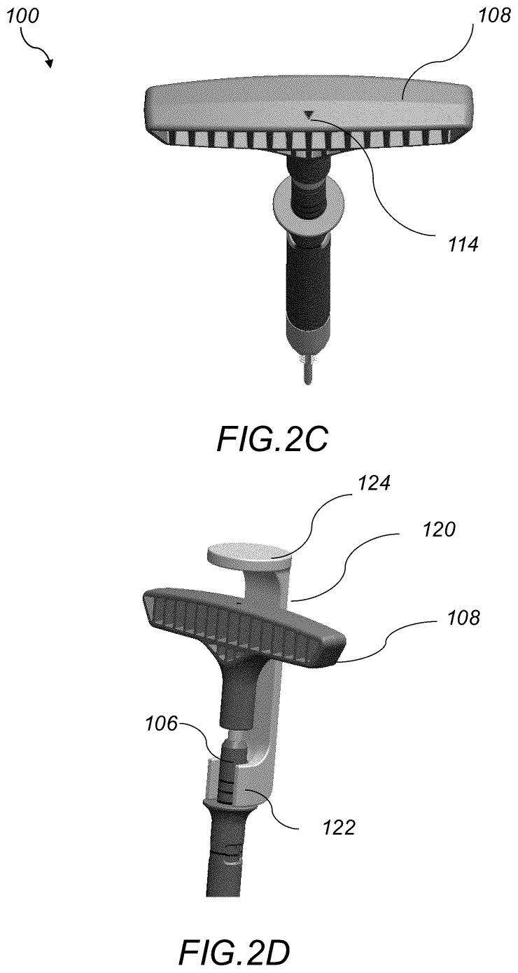

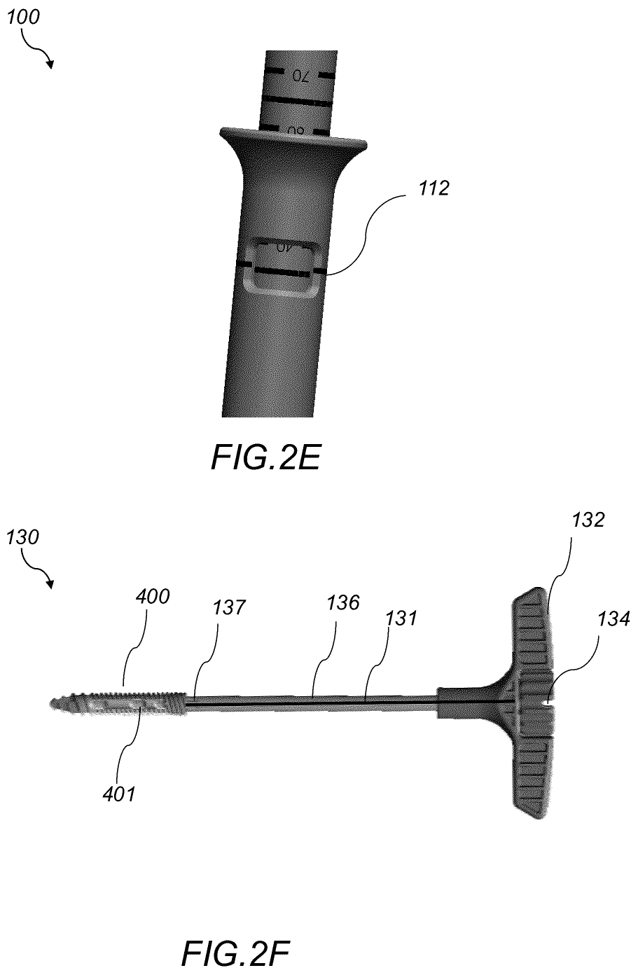

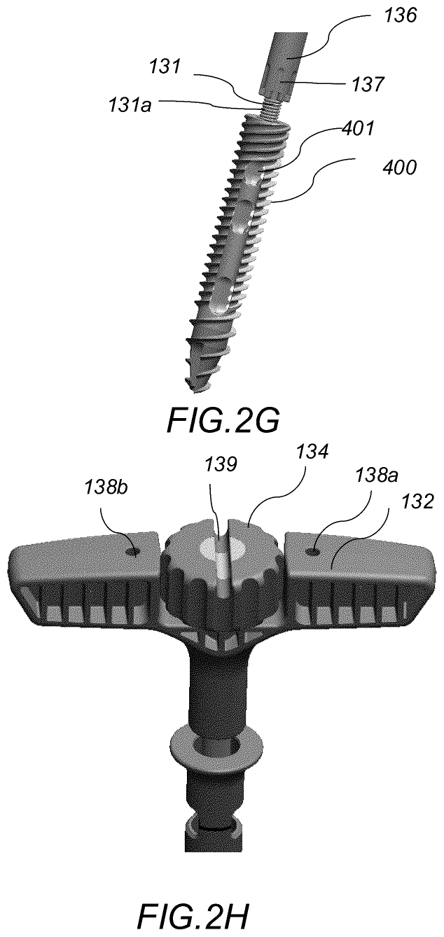

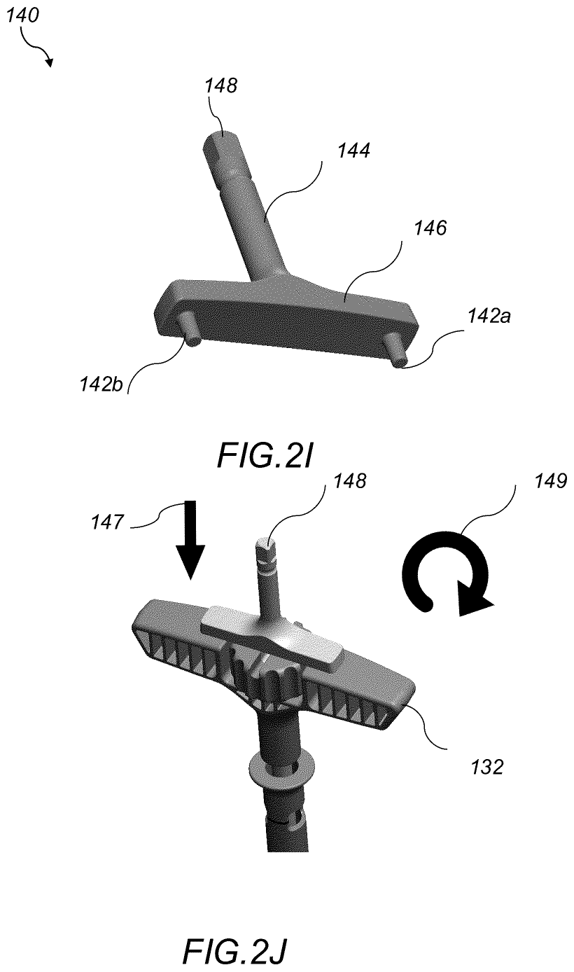

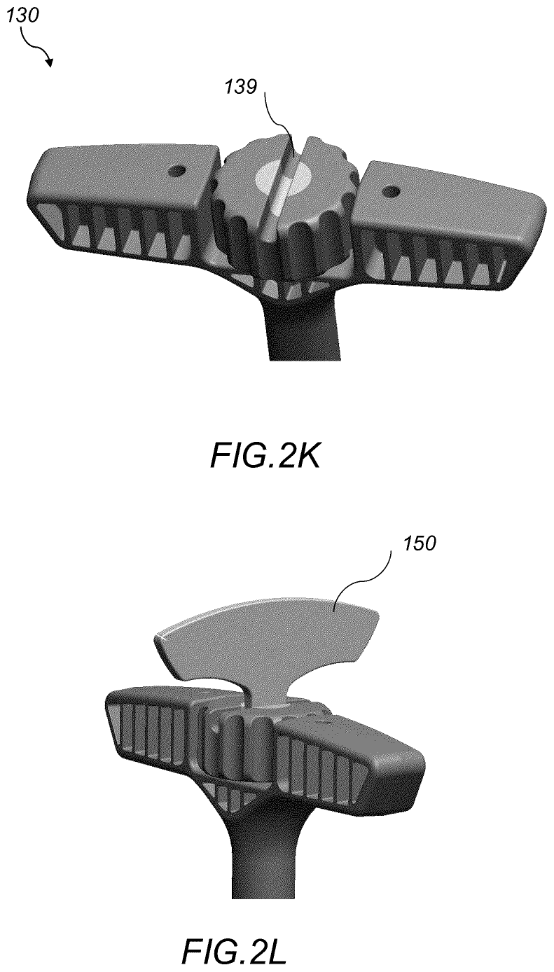

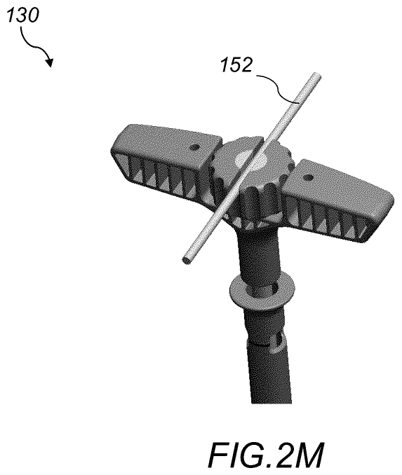

Referring to FIG. 2A-FIG. 2M, an awl-tap-dilator tool 100 that is used in the single step surgical technique of the present invention includes an elongated shaft 101 terminating at a beveled tip 102 at the distal end. Tip 102 serves as an awl that is used to pierce the bone and locate the appropriate trajectory under fluoroscopy. Tool 100 further includes a drill-tap section 104 adjacent to the tip 102, that is used to drill and tap the bone in order to form the bone openings 93a-93d. Tool 100 further includes a dilator section 106 that is adjacent to the drill-tap section 104, and surrounds and has a larger diameter than the elongated shaft 101. Dilator section 106 has markings 107 on its outside surface. Markings 107 are used to determine the implant length, as will be described below. Tool 100 further includes a handle 108 at the proximal end of the elongated shaft 101 that is used to assist in the insertion, and rotation of the tip, and drill sections of the tool. The dilator section 106 and the handle 108 are injection molded plastic, whereas the tip 102, the drill 104 and the shaft 101 are made of stainless steel. Tool 100 further includes a tissue protector 110 cannula that surrounds the dilator section 106. The tissue protector 110 includes a distal end 111 that is made of stainless steel and has teeth 113 at its front perimeter. Teeth 113 are impacted into the bone in order to maintain the tissue protector's 110 position. The rest of the body of the tissue protector 110 is injection molded plastic. Tissue protector 110 further includes a window 112 at the proximal end that has a line that lines up with the markings 107 of the dilator section 106 that indicate the implant length. The most proximal surface of the handle 108 includes an arrow 114 that functions as a directional marking indicating the orientation of the tip 102. The drill-tap portion 104 is initially advanced by impacting the proximal surface of the handle 108. The tissue protector 110 is then impacted into the bone so that the teeth 113 on its distal end 111 engage the bone and secure the position and trajectory of the tissue protector 110 and the tool 100 during the surgery. The tissue protector 110 is impacted into the bone using a tissue protector impacting component 120. Tissue protector impacting component 120 includes a flat surface 124 at the proximal end and a side slot opening 122 at the distal end, that is dimensioned to slide laterally over the proximal end of the dilator section 106 while the flat surface 124 is positioned over the tool handle 108. In operation, the side slot opening 122 of the tissue protector impacting component 120 slides over the proximal end of the dilator section 106 and the flat surface 124 of the proximal end of the tissue protector impacting component 120 is used to advance and impact the tissue protector 110 into the bone without changing the position and direction of the tool 100. Flat surface 124 may be impacted with a mallet or any other impacting tool. Tissue protector impacting component 120 is made of injection molded plastic or machined stainless steel. Once the tissue protector 110 is secured in place, the elongated shaft 101 with the dilator 106 are removed and an implant inserter tool 130 is used to insert implant screw 400 into any of the drilled openings 93a-93d through the tissue protector 110. Inserter tool 130 includes an elongated cannulated outer shaft 136 having a handle 132 at the proximal end and an inner stylet 131 extending through the cannula of the outer shaft 136. Inner stylet 131 has a distal end with threads 131a. Handle 132 includes a center cutout that holds a rotatable knob 134. Knob 134 is attached to the proximal end of the inner stylet 131. Implant screw 400 is attached to the distal end of the inner stylet 131 of the inserter tool 130 by aligning the positive tabs 137 at the distal end of the inserter tool with negative slots on the top of the implant 400 and then rotating knob 134 to engage the distal end threads 131a of the inner stylet 131 with inner threads in the top opening of the implant 400. The handle 132 and the knob 134 are made of injection molded plastic and the elongated shaft 136 is made of stainless steel. In cases where implant insertion under power is needed, a power adapter component 140 is used. Power adapter component 140 includes a shaft 144 and a coupling member 146 extending horizontally from the distal end of the shaft 144. The bottom surface of the coupling member 146 includes pins 142a, 142b that are shaped and dimensioned to fit within and engage openings 138a, 138b on the top surface of the handle 132, respectively. The proximal end 148 of shaft 144 is shaped and dimensioned to engage the tip of a power drill (not shown). The power drill is then used to rotate the handle 132 clockwise 149 and insert the implant into the bone under power. The handle 132 may also be impacted with a mallet or any other impacting tool to advance the implant in the direction 147. The handle 132 may also be rotated counter-clockwise to remove the implant 400, if necessary. Once the implant 400 is placed and secured in the appropriate bone opening, the inner stylet 131 is removed by rotating knob 134 counter-clockwise. The top of knob 134 includes a slot 139 that is shaped and dimensioned to receive an unlocking tool 150 or a rod 152, as shown in FIG. 2L and FIG. 2M, respectively. In one example, implant 400 is the bone fusing implant described in U.S. Pat. No. 12,064,154B2, and U.S. Pat. No. 9,717,538B2, the contents of which are incorporated herewith by reference.

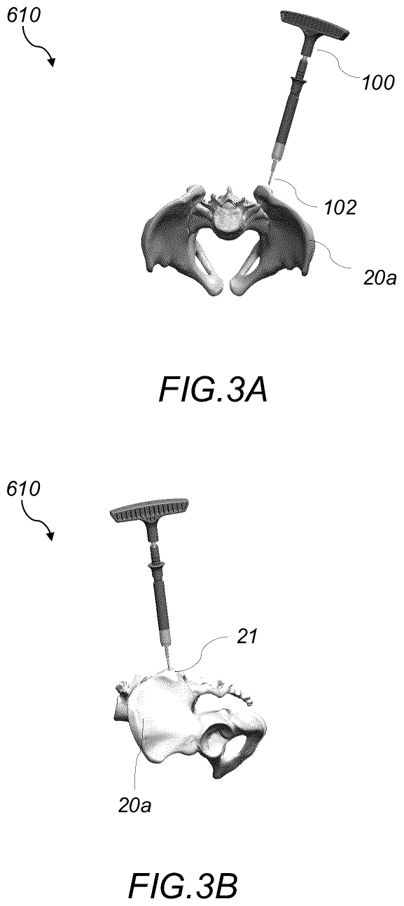

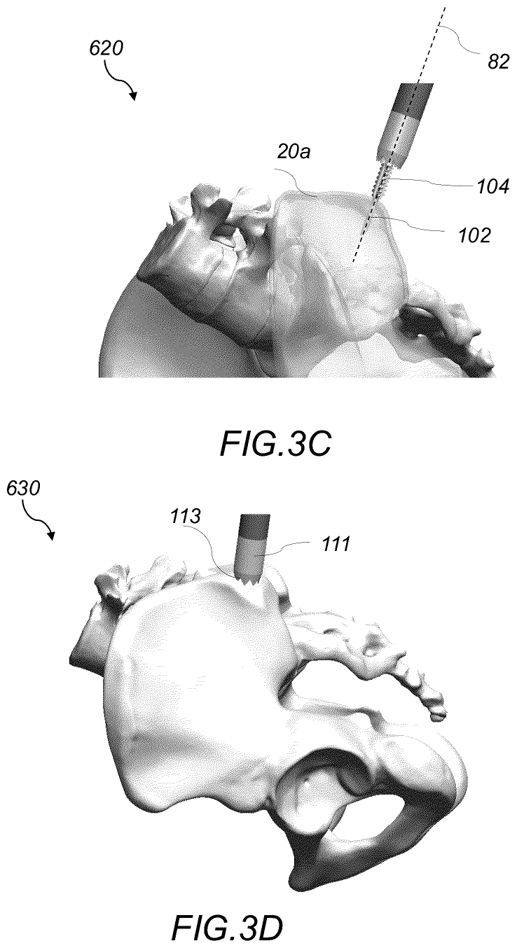

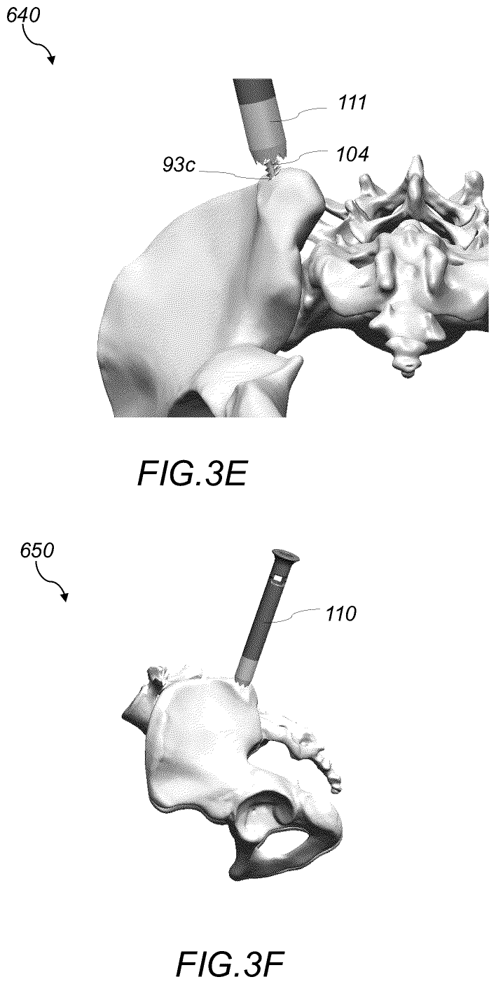

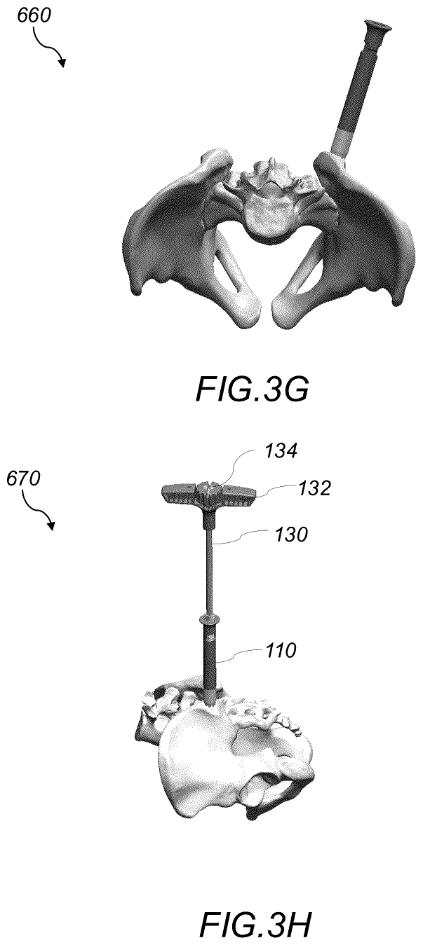

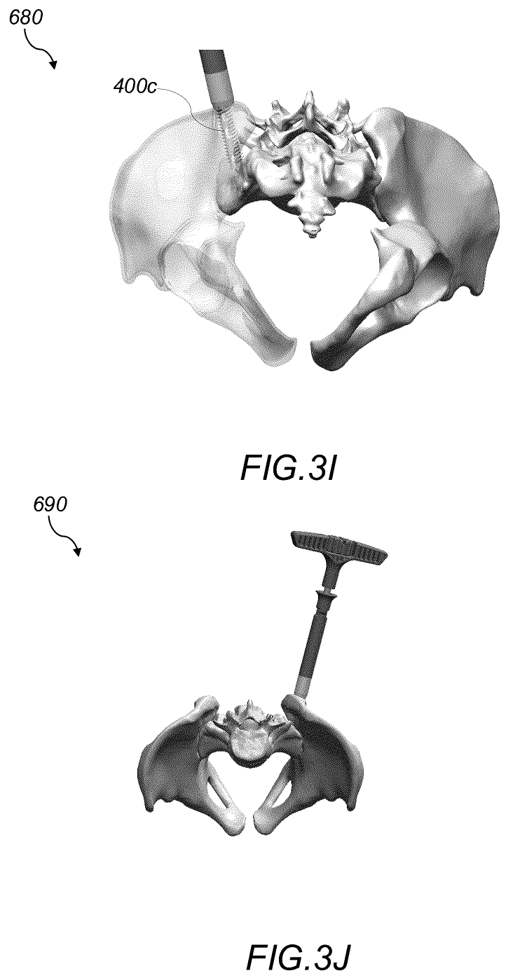

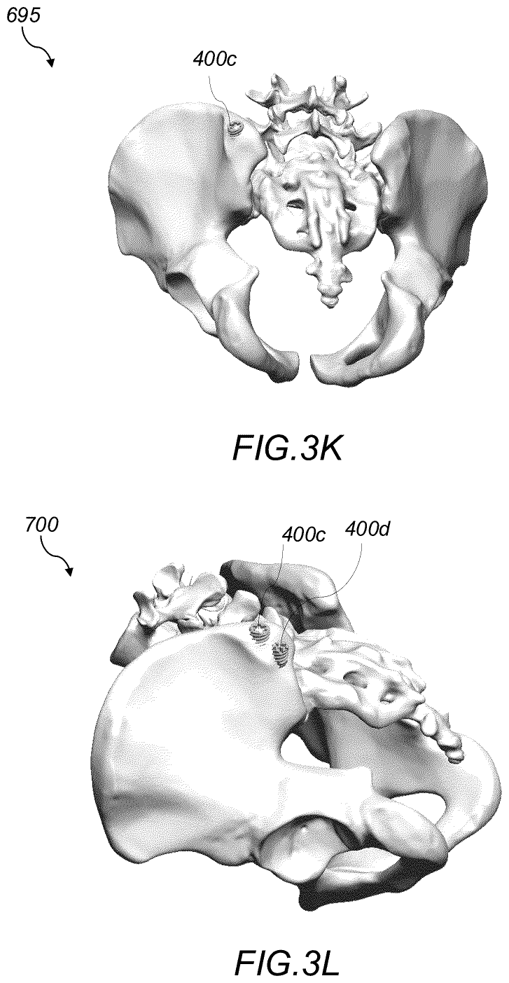

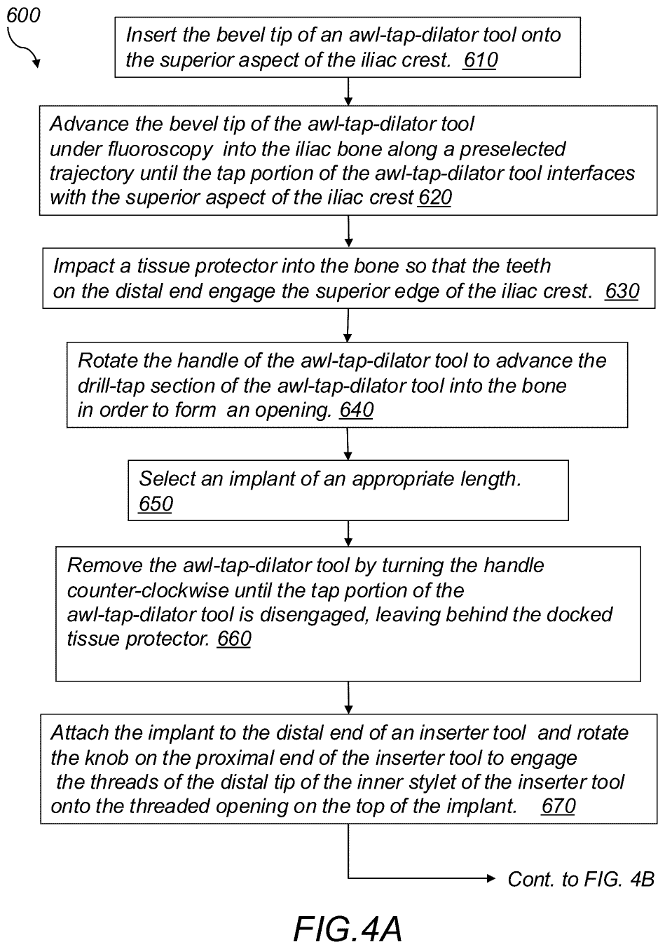

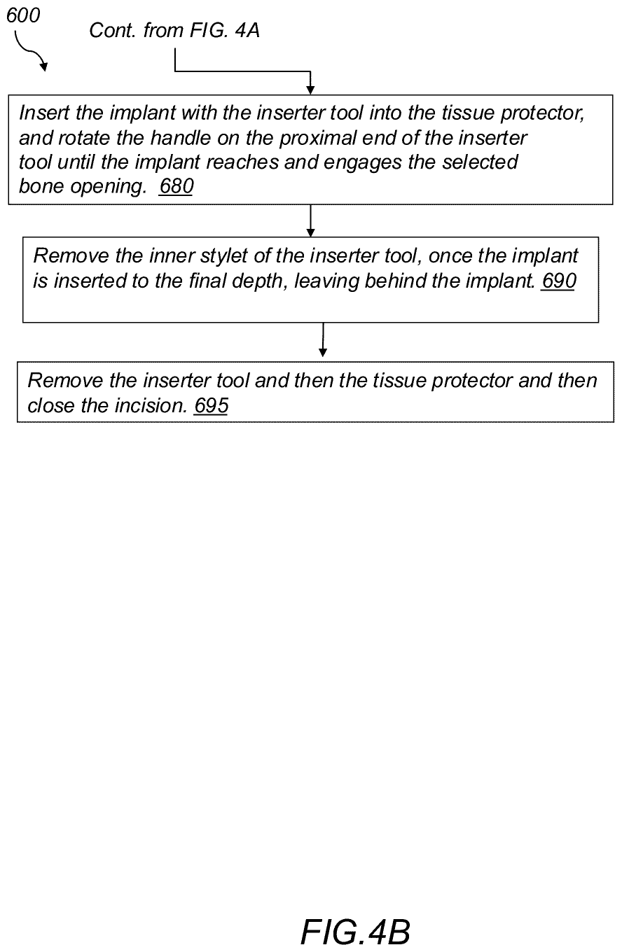

Referring to FIG. 3A-FIG. 4B, the process for targeting and fusing of the SI joint 600, according to this invention includes the following steps. First, the beveled tip 102 of the awl-tap-dilator tool 100 is inserted through a small incision on the skin of the patient and is docked onto the superior aspect of the iliac crest 21, as shown in FIG. 3A and FIG. 3B (610). The directional marking 114 on the handle 108 of the awl-tap-dilator tool 100 indicates the direction of the beveled tip 102. The beveled tip 102 is then advanced into the iliac bone 20a along a preselected trajectory 82 under fluoroscopic observation, until the tap portion 104 interfaces with the superior aspect of the iliac crest, as shown in FIG. 3C (620). Next, the initial advancement of the tap portion 104 is achieved by impacting the proximal surface of the handle 108. Next, the tissue protector 110 is impacted into the bone to ensure that the teeth 113 on the distal end 111 engage the superior edge of the iliac crest 20a, as shown in FIG. 3D (630). This step ensures that the selected trajectory 82 is maintained during the entire procedure. The tissue protector 110 is impacted using the tissue protector impacting instrument 120. For this purpose, the distal end 122 of the tissue protector impacting instrument 120 is slid laterally over the awl-tap-dilator tool shaft 106 and the proximal end 124 of the tissue protector impacting instrument 120 is slid over the handle 108, as shown in FIG. 2D. The proximal end 124 of the tissue protector impacting instrument 120 is then impacted with a mallet to drive the teeth 113 of the tissue protector distal end 111 into the bone 20a without changing the position and orientation of the awl-tap-dilator tool 100. Next, the tissue protector impacting instrument 120 is removed and the handle 108 of the awl-tap-dilator tool 100 is rotated to advance the drill-tap section 104 into the bone in order to form the opening 93c, as shown in FIG. 3E (640). Next, a screw implant 400c of an appropriate length is determined and selected (650). The appropriate length of the screw implant 400c is determined by comparing the marking lines 107 on the awl-tap-dilator tool 100 to the marking line 112 on the window of the tissue protector 110. Fluoroscopy is used to ensure proper screw length and trajectory. Once, the correct depth is achieved, the awl-tap-dilator tool 100 is removed by turning the handle 108 counter-clockwise until the tap portion 104 of the awl-tap-dilator tool 100 is disengaged. The docked tissue protector 110 cannula is left behind and the bone is prepared to receive the screw implant 400c, as shown in FIG. 3F (660). Next, the implant 400c is attached to the distal end of the inserter tool 130 by aligning the positive tabs 137 on the distal end of the inserter tool 130 to negative slots on the top of the implant 400c. The knob 134 on the proximal end of the inserter tool 130 is then rotated to engage the threads 131a of the distal tip of the inner stylet 131 of the inserter tool 130 onto the threaded opening on the top of the implant 400c (670). The implant 400c with the inserter tool 130 is then inserted into the tissue protector 110 cannula, as shown in FIG. 3H, and the handle 132 on the proximal end of the inserter tool 130 is rotated until the implant 400c screw reaches and engages the selected bone opening 93c, as shown in FIG. 3I (680). The insertion and placement of the implant 400c is monitored under fluoroscopy. If insertion under power is desired, the proximal end of the insertion tool handle 132 is attached to the power adapter component 140 and a power drill (not shown) is used to advance the implant 400c into the desired opening. Once, the implant 400c is inserted to the final depth, the inner stylet 131 is removed by turning the knob 134 on the inserter tool counter-clockwise, leaving behind the implant 400c (690). An unlocking tool 150, 152 may be used, if needed. Next, the inserter tool 130 is removed and then the tissue protector 110 cannula is removed, as shown in FIG. 3K (695). The implant placement procedure is repeated for placing additional implants 400a, 400b and 400d in other selected bone opening locations 93a, 93b, 93d, respectively, as shown in FIG. 3L and FIG. 1D (700).

Several embodiments of the present invention have been described. Nevertheless, it will be understood that various modifications may be made without departing from the spirit and scope of the invention. Accordingly, other embodiments are within the scope of the following claims.

What is claimed is:

1. An awl-tap-dilator tool used in a single step surgical technique, comprising:
an elongated shaft having a beveled tip at a distal end, a drill-tap section adjacent to the beveled tip, and a dilator section adjacent to the drill-tap section;
wherein said beveled tip is used for piercing a bone and locate an appropriate trajectory under fluoroscopy;
wherein said drill-tap section is used for drilling and tapping the bone in order to form a bone opening; and
wherein said dilator section surrounds the elongated shaft, has a larger diameter than the elongated shaft and is used for dilating tissue around and above the bone opening.

2. The awl-tap-dilator tool of claim 1, wherein said elongated shaft further comprises a handle at the proximal end of the elongated shaft and wherein said handle is used for inserting and rotating the beveled tip and drill-tap section.

3. The awl-tap-dilator tool of claim 2, wherein said handle comprises an arrow that functions as a directional marking indicating the beveled tip's orientation.

4. The awl-tap-dilator tool of claim 1, wherein said dilator section comprises markings on an outer surface that are used for determining length of an implant.

5. The awl-tap-dilator tool of claim 1, further comprising a tissue protector cannula that surrounds the dilator section.

6. The awl-tap-dilator tool of claim 5, wherein said tissue protector cannula comprises teeth at a distal end, and wherein said teeth are used for impacting the tissue protector into the bone around said bone opening.

7. The awl-tap-dilator tool of claim 5, wherein said tissue protector cannula comprises a window at a proximal end and has a marking line that is used for aligning with one of the markings of the dilator section in order to determine a length of an implant.

8. The awl-tap-dilator tool of claim 1, wherein said dilator section comprises injection molded plastic.

9. The awl-tap-dilator tool of claim 1, wherein said elongated shaft, said beveled tip, and said drill-tap section comprise stainless steel.

10. The awl-tap-dilator tool of claim 6, wherein said teeth of the tissue protector cannula comprise stainless steel and said tissue protector cannula comprises injection molded plastic.

11. The awl-tap-dilator tool of claim 6, further comprising a tissue protector impacting component and wherein said tissue protector impacting component includes a flat surface at a proximal end and a side slot opening at a distal end and wherein said side slot opening is shaped and sized to slide laterally over a proximal end of the dilator section while the flat surface of the proximal end of the tissue protector impacting component is used for advancing and impacting the tissue protector into the bone without changing position and direction of the awl-tap-dilator tool.

12. A system used in a single step surgical technique for forming an opening into a bone and inserting an implant into the bone opening comprising:
an awl-tap-dilator tool comprising:
an elongated shaft having a beveled tip at a distal end, a drill-tap section adjacent to the beveled tip, and a dilator section adjacent to the drill-tap section;
wherein said beveled tip is used for piercing a bone and locate an appropriate trajectory under fluoroscopy;
wherein said drill-tap section is used for drilling and tapping the bone in order to form the bone opening;
wherein said dilator section surrounds the elongated shaft, has a larger diameter than the elongated shaft and is used for dilating tissue around and above the bone opening;
a tissue protector cannula that surrounds the dilator section, and wherein said tissue protector cannula comprises teeth at a distal end, and wherein said teeth are used for impacting the tissue protector into the bone around said bone opening; and
an implant inserter tool used for inserting an implant into the bone opening through the tissue protector, wherein said inserter tool comprises an elongated cannulated outer shaft and an inner stylet extending through the outer shaft.

13. The system of claim 12, wherein the inserter tool comprises a handle at a proximal end and wherein said inner stylet comprises a rotatable knob at a proximal end and wherein said inserter tool handle comprises a center cutout shaped and sized to hold the rotatable knob.

14. The system of claim 13, wherein said inner stylet comprises a threaded distal end that is sized to engage threads on a proximal end of the implant.

15. The system of claim 13, further comprising a power adapter component used for attaching a power drill to the handle of the inserter tool.

16. The system of claim 15, wherein said power adapter component comprises a shaft and a coupling member and wherein the shaft has a proximal end shaped and sized to engage a power drill tip and said coupling member extends horizontally from a distal end of the shaft and wherein a bottom surface of the coupling member comprises pins that are shaped and sized to fit within and engage corresponding openings on a top surface of the handle.

17. A method for forming an opening into a bone and inserting an implant into the bone opening in a single step comprising:
providing an awl-tap-dilator tool comprising an elongated shaft having a beveled tip at a distal end, a drill-tap section adjacent to the beveled tip, a dilator section adjacent to the drill-tap section, and a tissue protector cannula that surrounds the dilator section;
piercing the bone with the beveled tip and locating an appropriate trajectory under fluoroscopy;
drilling and tapping the bone with the drill-tap section and forming the bone opening;
dilating tissue around and above said bone opening with the dilator section;
impacting the tissue protector cannula into the bone around said bone opening;
providing an implant inserter tool comprising an elongated cannulated outer shaft and an inner stylet extending through the outer shaft;
attaching the implant to a distal end of the inner stylet and inserting the implant inserter tool with the attached implant through the tissue protector; and
inserting the implant into the bone opening.

18. The method of claim 17, wherein said tissue protector cannula comprises a window at a proximal end and has a marking line that is used for aligning with a marking of the dilator section in order to determine a length of the implant.

19. The method of claim 17 wherein said tissue protector cannula comprises teeth at a distal end, and wherein said teeth are used for impacting the tissue protector into the bone around said bone opening.

20. The method of claim 19, further comprising providing a tissue protector impacting component and wherein said tissue protector impacting component includes a flat surface at a proximal end and a side slot opening at a distal end and wherein said side slot opening is shaped and sized to slide laterally over a proximal end of the dilator section while the flat surface of the proximal end of the tissue protector impacting component is used for advancing and impacting the tissue protector into the bone without changing position and direction of the awl-tap-dilator tool.
